SEQUENCE LISTING (2 sequences)

SEQ ID NO: 1 - DNA, 50 bp, Artificial Sequence
  16S_V3_F
  misc_feature (42): a or g or c or t/u
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc

(19) United States
(12) Patent Application Publication
(10) Pub. No.: US 2021/0189464 A1
(43) Pub. Date: Jun. 24, 2021

(54) METHOD FOR DIAGNOSING HEAD AND NECK CANCER VIA BACTERIAL METAGENOMIC ANALYSIS

(71) Applicant: MD HEALTHCARE INC., Mapo-gu, Seoul (KR)

(72) Inventor: Yoon-Keun KIM, Paju-si, Gyeonggi-do (KR)

(73) Assignee: MD HEALTHCARE INC., Seoul (KR)

(21) Appl. No.: 16/757,260

(22) PCT Filed: Apr. 26, 2018
(86) PCT No.: PCT/KR2018/004837

§ 371 Date: Apr. 17, 2020

(30) Foreign Application Priority Data
Oct. 18, 2017 (KR) 10-2017-0135467
Apr. 25, 2018 (KR) 10-2018-0047952

(51) Int. Cl. C12Q 1/689; C12Q 1/6886

(52) U.S. Cl. CPC C12Q 1/689; C12Q 1/686; C12Q 1/6886

(57) ABSTRACT

Provided is a method of diagnosing head and neck cancer by analyzing an increase or decrease in content of specific bacteria-derived extracellular vesicles through bacterial metagenomic analysis using normal individual- and subject-derived samples, wherein the risk of head and neck cancer can be diagnosed through metagenomic analysis of bacteria-derived extracellular vesicles using a human body-derived sample, and thus a risk group of head and neck cancer can be diagnosed early and predicted, thereby delaying the onset of head and neck cancer or preventing the onset of head and neck cancer through appropriate management, and even after head and neck cancer occur, early diagnosis for head and neck cancer can be implemented, thereby lowering the incidence of head and neck cancer and increasing therapeutic effects.

### TECHNICAL FIELD

The present invention relates to a method for diagnosing head and neck cancer through a bacterial metagenomic analysis and, more specifically, to a method of diagnosing head and neck cancer, and the like by performing a bacterial metagenomic analysis using normal individual-derived and subject-derived samples to analyze an increase or decrease in the content of specific bacteria-derived extracellular vesicles.

### BACKGROUND ART

The head and neck refer to parts from the brain to the upper chest and encompass the oral cavity, larynx, pharynx, nasal cavity, and the like, and cancers occurring in these organs are called head and neck cancer. Thereamong, oral cancer is cancer occurring in the oral cavity, which mainly originates in squamous cells constituting the mucous membrane surrounding the oral cavity. As risk factors for oral cancer, smoking, dirty oral hygiene, persistent chronic irritation, and the like are known. Pharyngeal cancer is a malignant tumor occurring in the pharyngeal mucous membrane, and smoking, drinking, viral infections, and the like are known as risk factors. According to data published by the Korea Central Cancer Registry, the annual number of patients with head and neck cancer except for thyroid cancer is 3,000, and the incidence frequency thereof ranks eighth among cancers. The diagnosis of head and neck cancer is determined by biopsy by removing tissue from tumors when cancer is suspected. In addition, to determine the extent of cancer, tests such as computed tomography, magnetic resonance imaging examination, and positron emission tomography are performed.

Meanwhile, it is known that the number of microorganisms symbiotically living in the human body is 100 trillion which is 10 times the number of human cells, and the number of genes of microorganisms exceeds 100 times the number of human genes. A microbiota is a microbial community that includes bacteria, archaea, and eukaryotes present in a given habitat. The intestinal microbiota is known to play a vital role in human's physiological phenomena and significantly affect human health and diseases through interactions with human cells. Bacteria coexisting in human bodies secrete nanometer-sized vesicles to exchange information about genes, proteins, low molecular weight compound, and the like with other cells. The mucous membranes form a physical barrier membrane that does not allow particles with the size of 200 nm or more to pass therethrough, and thus bacteria symbiotically living in the mucous membranes are unable to pass therethrough, but bacteria-derived extracellular vesicles have a size of approximately 100 nm or less and thus relatively freely pass through the mucous membranes and are absorbed into the human body.

Metagenomics, also called environmental genomics, may be analytics for metagenomic data obtained from samples collected from the environment (Korean Patent Publication No. 2011-073049). Recently, the bacterial composition of human microbiota has been listed using a method based on 16s ribosomal RNA (16s rRNA) base sequences, and 16s rDNA base sequences, which are genes of 16s ribosomal RNA, are analyzed using a next generation sequencing (NGS) platform. However, as for the occurrence of head and neck cancer, there is no report about a method of identifying, from a human-derived material such as saliva, a causative factor of head and neck cancer by analysis of metagenomes present in bacteria-derived vesicles and of diagnosing head and neck cancer.

### DISCLOSURE

### Technical Problem

The present inventors extracted genes from bacteria-derived extracellular vesicles present in saliva as normal individual-derived and subject-derived samples and performed a metagenomic analysis in this regard in order to diagnose the causal factors and risk of head and neck cancer in advance, and as a result, identified bacteria-derived extracellular vesicles which may act as a causal factor of head and neck cancer, thereby completing the present invention based on this.

Therefore, an object of the present invention is to provide a method of providing information for diagnosing head and neck cancer, a method of diagnosing head and neck cancer, a method of predicting the risk of head and neck cancer onset, and the like through the metagenomic analysis of bacteria-derived extracellular vesicles.

However, the technical goals of the present invention are not limited to the aforementioned goals, and other unmentioned technical goals will be clearly understood by those of ordinary skill in the art from the following description.

### Technical Solution

To achieve the above-described object of the present invention, there is provided a method of providing information for head and neck cancer diagnosis, comprising the following processes:

(a) extracting DNAs from extracellular vesicles isolated from normal individual and subject samples;

(b) performing polymerase chain reaction (PCR) on the extracted DNA using a pair of primers comprising SEQ ID NO: 1 and SEQ ID NO: 2; and

(c) comparing an increase or decrease in content of bacteria-derived extracellular vesicles of the subject-derived sample with that of a normal individual-derived sample through sequencing of a product of the PCR.

The present invention also provides a method of diagnosing head and neck cancer, comprising the following processes:

(a) extracting DNAs from extracellular vesicles isolated from normal individual and subject samples;

(b) performing polymerase chain reaction (PCR) on the extracted DNA using a pair of primers comprising SEQ ID NO: 1 and SEQ ID NO: 2; and

(c) comparing an increase or decrease in content of bacteria-derived extracellular vesicles of the subject-derived sample with that of a normal individual-derived sample through sequencing of a product of the PCR.

The present invention also provides a method of predicting a risk for head and neck cancer, comprising the following processes:

(a) extracting DNAs from extracellular vesicles isolated from normal individual and subject samples;

(b) performing polymerase chain reaction (PCR) on the extracted DNA using a pair of primers comprising SEQ ID NO: 1 and SEQ ID NO: 2; and

(c) comparing an increase or decrease in content of bacteria-derived extracellular vesicles of the subject-derived sample with that of a normal individual-derived sample through sequencing of a product of the PCR.

In one embodiment of the present invention, the sample may be saliva.

In another embodiment of the present invention, in process (c), the head and neck cancer may be diagnosed by comparing an increase or decrease in content of extracellular vesicles derived from one or more bacteria selected from the group consisting of the phylum Cyanobacteria and the phylum Fusobacteria.

In another embodiment of the present invention, in process (c), the head and neck cancer may be diagnosed by comparing an increase or decrease in content of extracellular vesicles derived from one or more bacteria selected from the group consisting of the class Halobacteria, the class Chloroplast, the class Fusobacteriia, and the class Epsilonproteobacteria.

In another embodiment of the present invention, in process (c), the head and neck cancer may be diagnosed by comparing an increase or decrease in content of extracellular vesicles derived from one or more bacteria selected from the group consisting of the order Halobacteriales, the order Bifidobacteriales, the order Streptophyta, the order Pseudomonadales, the order Oceanospirillales, the order Fusobacteriales, and the order Campylobacterales.

In another embodiment of the present invention, in process (c), the head and neck cancer may be diagnosed by comparing an increase or decrease in content of extracellular vesicles derived from one or more bacteria selected from the group consisting of the family Pseudomonadaceae, the family Halobacteriaceae, the family Oxalobacteraceae, the family Halomonadaceae, the family Comamonadaceae, the family Lactobacillaceae, the family Paraprevotellaceae, the family Fusobacteriaceae, and the family Campylobacteraceae.

In another embodiment of the present invention, in process (c), the head and neck cancer may be diagnosed by comparing an increase or decrease in content of extracellular vesicles derived from one or more bacteria selected from the group consisting of the genus Cupriavidus, the genus Chromohalobacter, the genus Pseudomonas, the genus Acinetobacter, the genus Enhydrobacter, the genus Lactobacillus, the genus Veillonella, the genus Fusobacterium, the genus Actinomyces, the genus Prevotella, the genus Megasphaera, the genus Campylobacter, and the genus Oribacterium.

In another embodiment of the present invention, process (c) may comprise comparing an increase or decrease in content of extracellular vesicles derived from one or more bacteria selected from the group consisting of the phylum Cyanobacteria and the phylum Fusobacteria;

extracellular vesicles derived from one or more bacteria selected from the group consisting of the class Halobacteria, the class Chloroplast, the class Fusobacteriia, and the class Epsilonproteobacteria;

extracellular vesicles derived from one or more bacteria selected from the group consisting of the order Halobacteriales, the order Bifidobacteriales, the order Streptophyta, the order Pseudomonadales, the order Oceanospirillales, the order Fusobacteriales, and the order Campylobacterales;

extracellular vesicles derived from one or more bacteria selected from the group consisting of the family Pseudomonadaceae, the family Halobacteriaceae, the family Oxalobacteraceae, the family Halomonadaceae, the family Comamonadaceae, the family Lactobacillaceae, the family Paraprevotellaceae, the family Fusobacteriaceae, and the family Campylobacteraceae; or

extracellular vesicles derived from one or more bacteria selected from the group consisting of the genus Cupriavidus, the genus Chromohalobacter, the genus Pseudomonas, the genus Acinetobacter, the genus Enhydrobacter, the genus Lactobacillus, the genus Veillonella, the genus Fusobacterium, the genus Actinomyces, the genus Prevotella, the genus Megasphaera, the genus Campylobacter, and the genus Oribacterium.

In another embodiment of the present invention, in process (c), in comparison with the normal individual-derived sample, it is possible to diagnose an increase in the content of the following as head and neck cancer:

extracellular vesicles derived from bacteria of the phylum Fusobacteria,

extracellular vesicles derived from one or more bacteria selected from the group consisting of the class Fusobacteriia and the class Epsilonproteobacteria, extracellular vesicles derived from one or more bacteria selected from the group consisting of the order Fusobacteriales and the order Campylobacterales,

extracellular vesicles derived from one or more bacteria selected from the group consisting of the family Lactobacillaceae, the family Paraprevotellaceae, the family Fusobacteriaceae, and the family Campylobacteraceae, or

extracellular vesicles derived from one or more bacteria selected from the group consisting of the genus Lactobacillus, the genus Veillonella, the genus Fusobacterium, the genus Actinomyces, the genus Prevotella, the genus Megasphaera, the genus Campylobacter, and the genus Oribacterium.

In another embodiment of the present invention, in process (c), in comparison with the normal individual-derived sample, it is possible to diagnose a decrease in the content of the following as head and neck cancer:

extracellular vesicles derived from bacteria of the phylum Cyanobacteria,

extracellular vesicles derived from one or more bacteria selected from the group consisting of the class Halobacteria and the class Chloroplast,

extracellular vesicles derived from one or more bacteria selected from the group consisting of the order Halobacteriales, the order Bifidobacteriales, the order Streptophyta, the order Pseudomonadales, and the order Oceanospirillales,

extracellular vesicles derived from one or more bacteria selected from the group consisting of the family Pseudomonadaceae, the family Halobacteriaceae, the family Oxalobacteraceae, the family Halomonadaceae, and the family Comamonadaceae, or

extracellular vesicles derived from one or more bacteria selected from the group consisting of the genus Cupriavidus, the genus Chromohalobacter, the genus Pseudomonas, the genus Acinetobacter, and the genus Enhydrobacter.

### Advantageous Effects

Extracellular vesicles secreted from bacteria present in the environment are absorbed into the human body, and thus may directly affect the occurrence of cancer, and it is difficult to diagnose head and neck cancer early before the onset of symptoms, and thus effective treatment thereof is difficult. Thus, according to the present invention, the causative factors and risk of head and neck cancer can be diagnosed through metagenomic analysis of bacteria-derived extracellular vesicles using a human body-derived sample, and thus a risk group of head and neck cancer can be diagnosed early, thereby delaying the onset of head and neck cancer or preventing the onset of head and neck cancer through appropriate management. In addition, even after head and neck cancer occurs, early diagnosis for head and neck cancer can be implemented, thereby lowering the incidence of head and neck cancer and increasing therapeutic effects, and the metagenomic analysis enables patients diagnosed with head and neck cancer to avoid exposure to causative factors predicted thereby, whereby the progression of the cancer is ameliorated, or the recurrence of head and neck cancer can be prevented.

### DESCRIPTION OF DRAWINGS

FIG. 1A illustrates images showing the distribution pattern of bacteria and extracellular vesicles over time after intestinal bacteria and bacteria-derived extracellular vesicles (EVs) were orally administered to mice, and FIG. 1B illustrates images showing the distribution pattern of bacteria and EVs after being orally administered to mice and, at 12 hours, saliva and various organs were extracted.

FIG. 2 is a result showing the distribution of bacteria-derived extracellular vesicles (EVs), which is significant in diagnostic performance at the phylum level by isolating bacteria-derived vesicles from saliva of a patient with head and neck cancer and a normal individual, and then performing a metagenomic analysis.

FIG. 3 is a result showing the distribution of bacteria-derived extracellular vesicles (EVs), which is significant in diagnostic performance at the class level by isolating bacteria-derived vesicles from saliva of a patient with head and neck cancer and a normal individual, and then performing a metagenomic analysis.

FIG. 4 is a result showing the distribution of bacteria-derived extracellular vesicles (EVs), which is significant in diagnostic performance at the order level by isolating bacteria-derived vesicles from saliva of a patient with head and neck cancer and a normal individual, and then performing a metagenomic analysis.

FIG. 5 is a result showing the distribution of bacteria-derived extracellular vesicles (EVs), which is significant in diagnostic performance at the family level by isolating bacteria-derived vesicles from saliva of a patient with head and neck cancer and a normal individual, and then performing a metagenomic analysis.

FIG. 6 is a result showing the distribution of bacteria-derived extracellular vesicles (EVs), which is significant in diagnostic performance at the genus level by isolating bacteria-derived vesicles from saliva of a patient with head and neck cancer and a normal individual, and then performing a metagenomic analysis.

### BEST MODE

The present invention relates to a method of diagnosing head and neck cancer through bacterial metagenomic analysis. The inventors of the present invention extracted genes from bacteria-derived extracellular vesicles using a normal individual and a subject-derived sample, performed metagenomic analysis thereon, and identified bacteria-derived extracellular vesicles capable of acting as a causative factor of head and neck cancer.

Therefore, the present invention provides a method of providing information for diagnosing head and neck cancer, the method comprising:

(a) extracting DNAs from extracellular vesicles isolated from normal individual and subject samples;

(b) performing polymerase chain reaction (PCR) on the extracted DNA using a pair of primers comprising SEQ ID NO: 1 and SEQ ID NO: 2; and

(c) comparing an increase or decrease in content of bacteria-derived extracellular vesicles of the subject-derived sample with that of a normal individual-derived sample through sequencing of a product of the PCR.

The term “head and neck cancer” as used herein refers to cancers occurring in organs such as the oral cavity, larynx, pharynx, nasal cavity, and encompasses oral cancer, salivary gland cancer, pharyngeal cancer, and nasal cavity cancer.

The term “head and neck cancer diagnosis” as used herein refers to determining whether a patient has a risk for head and neck cancer, whether the risk for head and neck cancer is relatively high, or whether head and neck cancer has already occurred. The method of the present invention may be used to delay the onset of head and neck cancer through special and appropriate care for a specific patient, which is a patient having a high risk for head and neck cancer or prevent the onset of head and neck cancer. In addition, the method may be clinically used to determine treatment by selecting the most appropriate treatment method through early diagnosis of head and neck cancer.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, bacterial metagenomic analysis is performed using bacteria-derived extracellular vesicles isolated from, for example, serum.

In the present invention, the normal individual and subject samples may be saliva, but the present invention is not limited thereto.

In an embodiment of the present invention, metagenomic analysis is performed on the bacteria-derived extracellular vesicles, and bacteria-derived extracellular vesicles capable of acting as a cause of the onset of head and neck cancer were actually identified by analysis at phylum, class, order, family, and genus levels.

More particularly, in one embodiment of the present invention, as a result of performing bacterial metagenomic analysis on extracellular vesicles present in subject-derived saliva samples at a phylum level, the content of extracellular vesicles derived from bacteria belonging to the phylum Cyanobacteria and the phylum Fusobacteria was significantly different between head and neck cancer patients and normal individuals (see Example 4).

More particularly, in one embodiment of the present invention, as a result of performing bacterial metagenomic analysis on extracellular vesicles present in subject-derived saliva samples at a class level, the content of extracellular vesicles derived from bacteria belonging to the class Halobacteria, the class Chloroplast, the class Fusobacteriia, and the class Epsilonproteobacteria was significantly different between head and neck cancer patients and normal individuals (see Example 4).

More particularly, in one embodiment of the present invention, as a result of performing bacterial metagenomic analysis on extracellular vesicles present in subject-derived saliva samples at an order level, the content of extracellular vesicles derived from bacteria belonging to the order Halobacteriales, the order Bifidobacteriales, the order Streptophyta, the order Pseudomonadales, the order Oceanospirillales, the order Fusobacteriales, and the order Campylobacterales was significantly different between head and neck cancer patients and normal individuals (see Example 4).

More particularly, in one embodiment of the present invention, as a result of performing bacterial metagenomic analysis on extracellular vesicles present in subject-derived saliva samples at a family level, the content of extracellular vesicles derived from bacteria belonging to the family Pseudomonadaceae, the family Halobacteriaceae, the family Oxalobacteraceae, the family Halomonadaceae, the family Comamonadaceae, the family Lactobacillaceae, the family Paraprevotellaceae, the family Fusobacteriaceae, and the family Campylobacteraceae was significantly different between head and neck cancer patients and normal individuals (see Example 4).

More particularly, in one embodiment of the present invention, as a result of performing bacterial metagenomic analysis on extracellular vesicles present in subject-derived saliva samples at a genus level, the content of extracellular vesicles derived from bacteria belonging to the genus Cupriavidus, the genus Chromohalobacter, the genus Pseudomonas, the genus Acinetobacter, the genus Enhydrobacter, the genus Lactobacillus, the genus Veillonella, the genus Fusobacterium, the genus Actinomyces, the genus Prevotella, the genus Megasphaera, the genus Campylobacter, and the genus Oribacterium was significantly different between head and neck cancer patients and normal individuals (see Example 4).

Through the results of the examples, it was confirmed that distribution variables of the identified bacteria-derived extracellular vesicles could be usefully used for the prediction of the onset of head and neck cancer.

### MODE OF THE INVENTION

Hereinafter, the present invention will be described with reference to exemplary examples to aid in understanding of the present invention. However, these examples are provided only for illustrative purposes and are not intended to limit the scope of the present invention.

### EXAMPLES

### Example 1. Analysis of In Vivo Absorption, Distribution, and Excretion Patterns of Bacteria and Bacteria-Derived Extracellular Vesicles

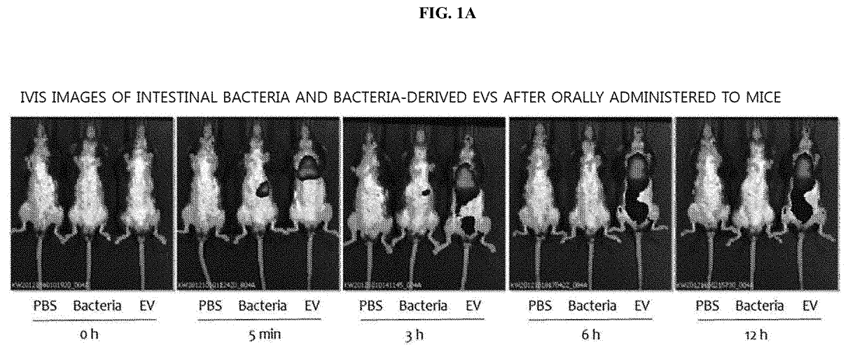

To evaluate whether bacteria and bacteria-derived vesicles are absorbed systemically through the mucosa, an experiment was performed using the following method. More particularly, 50 μg of each of bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After bacteria and bacteria-derived extracellular vesicles were systematically absorbed, to evaluate a pattern of invasion of intestinal bacteria and the bacteria-derived EVs into various organs in the human body after being systematically absorbed, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the bacteria-derived EVs were distributed in the blood, heart, lungs, liver, kidneys, spleen, adipose tissue, and muscle.

### Example 2. Vesicle Isolation and DNA Extraction from Saliva

To isolate vesicles and extract DNA, from saliva, first, saliva was added to a 10 ml tube and centrifuged at 3,500×g and 4° C. for 10 min to precipitate a suspension, and only a supernatant was collected, which was then placed in a new 10 ml tube. The collected supernatant was filtered using a 0.22 μm filter to remove bacteria and impurities, and then placed in centrifugal filters (50 kD) and centrifuged at 1500×g and 4° C. for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4° C. for 3 hours by using a Type 90ti rotor to remove a supernatant, and the agglomerated pellet was dissolved with phosphate-buffered saline (PBS), thereby obtaining vesicles.

100 μl of the extracellular vesicles isolated from the saliva according to the above-described method was boiled at 100° C. to allow the internal DNA to come out of the lipid and then cooled on ice for 5 minutes. Next, the resulting vesicles were centrifuged at 10,000×g and 4° C. for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16s rDNA primers shown in Table 1 below.

TABLE 1

| Primer | | Sequence | SEQ ID NO. |
|---|---|---|---|
| 16S rDNA | 16S_V3_F | 5′-TCGTCGGCAGCGTC | 1 |
| | | AGATGTGTATAAGAG | |
| | | ACAGCCTACGGGNGG | |
| | | CWGCAG-3′ | |
| | 16S_V4_R | 5′-GTCTCGTGGGCTCG | 2 |
| | | GAGATGTGTATAAGA | |
| | | GACAGGACTACHVGG | |
| | | GTATCTAATCC-3′ | |

### Example 3. Metagenomic Analysis Using DNA Extracted from Saliva

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score <20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4. Head and Neck Cancer Diagnostic Model Based on Metagenomic Analysis of Bacteria-Derived EVs Isolated from Saliva

EVs were isolated from saliva samples of 50 head and neck cancer patients and 215 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), accuracy, sensitivity, and specificity, which are diagnostic performance indexes, were calculated by logistic regression analysis.

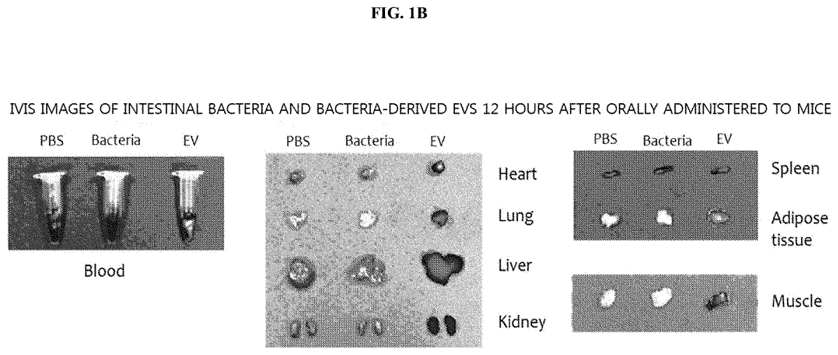

As a result of analyzing bacteria-derived EVs in saliva at a phylum level, a diagnostic model developed using bacteria belonging to the phylum Cyanobacteria and the phylum Fusobacteria as a biomarker exhibited significant diagnostic performance for head and neck cancer (see Table 2 and FIG. 2).

TABLE 2

| | | | Head and | | | | | | | |
|---|---|---|---|---|---|---|---|---|---|---|
| | Control | | Neck Cancer | | t-test | | | | | |
| Taxon | Mean | SD | Mean | SD | p-value | Ratio | AUC | Accuracy | sensitivity | specificity |
| p_Cyanobacteria | 0.0202 | 0.0322 | 0.0068 | 0.0081 | 0.0000 | 0.33 | 0.72 | 0.82 | 1.00 | 0.02 |
| p_Fusobacteria | 0.0097 | 0.0140 | 0.0235 | 0.0296 | 0.0025 | 2.42 | 0.69 | 0.83 | 0.99 | 0.12 |

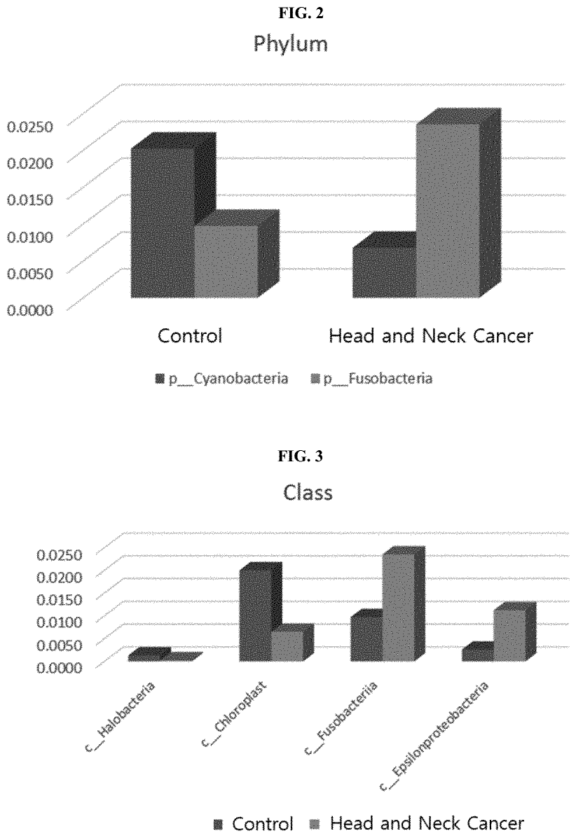

As a result of analyzing bacteria-derived EVs in saliva at a class level, a diagnostic model developed using bacteria belonging to the class Halobacteria, the class Chloroplast, the class Fusobacteriia, and the class Epsilonproteobacteria as a biomarker exhibited significant diagnostic performance for head and neck cancer (see Table 3 and FIG. 3).

TABLE 3

| | | | Head and | | | | | | | |
|---|---|---|---|---|---|---|---|---|---|---|
| | Control | | Neck Cancer | | t-test | | | | | |
| Taxon | Mean | SD | Mean | SD | p-value | Ratio | AUC | Accuracy | sensitivity | specificity |
| c_Halobacteria | 0.0013 | 0.0035 | 0.0002 | 0.0004 | 0.0000 | 0.18 | 0.72 | 0.81 | 1.00 | 0.02 |
| c_Chloroplast | 0.0200 | 0.0321 | 0.0065 | 0.0080 | 0.0000 | 0.32 | 0.73 | 0.82 | 1.00 | 0.02 |
| c_Fusobacteriia | 0.0097 | 0.0140 | 0.0235 | 0.0296 | 0.0025 | 2.42 | 0.69 | 0.83 | 0.99 | 0.12 |
| c_Epsilonproteobacteria | 0.0026 | 0.0046 | 0.0112 | 0.0200 | 0.0041 | 4.36 | 0.74 | 0.84 | 0.99 | 0.20 |

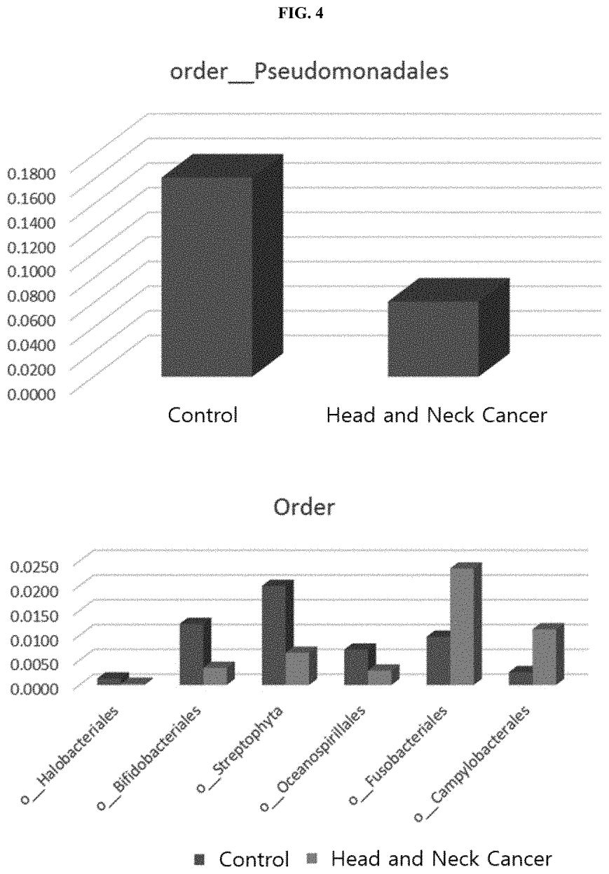

As a result of analyzing bacteria-derived EVs in saliva at an order level, a diagnostic model developed using bacteria belonging to the order Halobacteriales, the order Bifidobacteriales, the order Streptophyta, the order Pseudomonadales, the order Oceanospirillales, the order Fusobacteriales, and the order Campylobacterales as a biomarker exhibited significant diagnostic performance for head and neck cancer (see Table 4 and FIG. 4).

TABLE 4

| | | | Head and | | | | | | | |
|---|---|---|---|---|---|---|---|---|---|---|
| | Control | | Neck Cancer | | t-test | | | | | |
| Taxon | Mean | SD | Mean | SD | p-value | Ratio | AUC | Accuracy | sensitivity | specificity |
| o_Halobacteriales | 0.0013 | 0.0035 | 0.0002 | 0.0004 | 0.0000 | 0.18 | 0.72 | 0.81 | 1.00 | 0.02 |
| o_Bifidobacteriales | 0.0123 | 0.0198 | 0.0034 | 0.0032 | 0.0000 | 0.28 | 0.70 | 0.81 | 1.00 | 0.00 |
| o_Streptophyta | 0.0200 | 0.0321 | 0.0065 | 0.0080 | 0.0000 | 0.32 | 0.73 | 0.82 | 1.00 | 0.02 |
| o_Pseudomonadales | 0.1615 | 0.1683 | 0.0609 | 0.0819 | 0.0000 | 0.38 | 0.78 | 0.83 | 0.98 | 0.18 |
| o_Oceanospirillales | 0.0071 | 0.0081 | 0.0029 | 0.0029 | 0.0000 | 0.41 | 0.74 | 0.82 | 1.00 | 0.04 |
| o_Fusobacteriales | 0.0097 | 0.0140 | 0.0235 | 0.0296 | 0.0025 | 2.42 | 0.69 | 0.83 | 0.99 | 0.12 |
| o_Campylobacterales | 0.0026 | 0.0046 | 0.0112 | 0.0200 | 0.0041 | 4.36 | 0.74 | 0.84 | 0.99 | 0.20 |

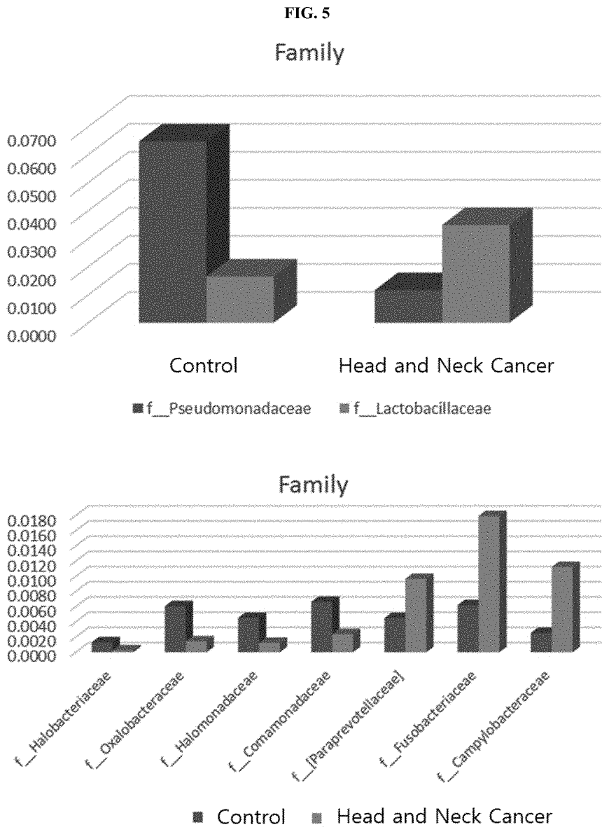

As a result of analyzing bacteria-derived EVs in saliva at a family level, a diagnostic model developed using bacteria belonging to the family Pseudomonadaceae, the family Halobacteriaceae, the family Oxalobacteraceae, the family Halomonadaceae, the family Comamonadaceae, the family Lactobacillaceae, the family Paraprevotellaceae, the family Fusobacteriaceae, and the family Campylobacteraceae as a biomarker exhibited significant diagnostic performance for head and neck cancer (see Table 5 and FIG. 5).

TABLE 5

| | | | Head and | | | | | | | |
|---|---|---|---|---|---|---|---|---|---|---|
| | Control | | Neck Cancer | | t-test | | | | | |
| Taxon | Mean | SD | Mean | SD | p-value | Ratio | AUC | Accuracy | sensitivity | specificity |
| f_Pseudomonadaceae | 0.0646 | 0.1537 | 0.0116 | 0.0124 | 0.0000 | 0.18 | 0.76 | 0.83 | 1.00 | 0.08 |
| f_Halobacteriaceae | 0.0013 | 0.0035 | 0.0002 | 0.0004 | 0.0000 | 0.18 | 0.72 | 0.81 | 1.00 | 0.02 |
| f_Oxalobacteraceae | 0.0060 | 0.0190 | 0.0014 | 0.0024 | 0.0007 | 0.23 | 0.71 | 0.81 | 0.99 | 0.02 |
| f_Halomonadaceae | 0.0045 | 0.0062 | 0.0012 | 0.0019 | 0.0000 | 0.27 | 0.79 | 0.82 | 0.98 | 0.12 |
| f_Comamonadaceae | 0.0066 | 0.0133 | 0.0024 | 0.0027 | 0.0000 | 0.36 | 0.72 | 0.81 | 1.00 | 0.02 |
| f_Lactobacillaceae | 0.0164 | 0.0156 | 0.0348 | 0.0451 | 0.0069 | 2.12 | 0.75 | 0.82 | 0.97 | 0.14 |
| f_[Paraprevotellaceae] | 0.0045 | 0.0054 | 0.0096 | 0.0093 | 0.0004 | 2.15 | 0.74 | 0.82 | 0.98 | 0.16 |
| f_Fusobacteriaceae | 0.0062 | 0.0096 | 0.0179 | 0.0273 | 0.0045 | 2.90 | 0.68 | 0.83 | 0.99 | 0.14 |
| f_Campylobacteraceae | 0.0025 | 0.0046 | 0.0112 | 0.0200 | 0.0040 | 4.43 | 0.74 | 0.84 | 0.99 | 0.20 |

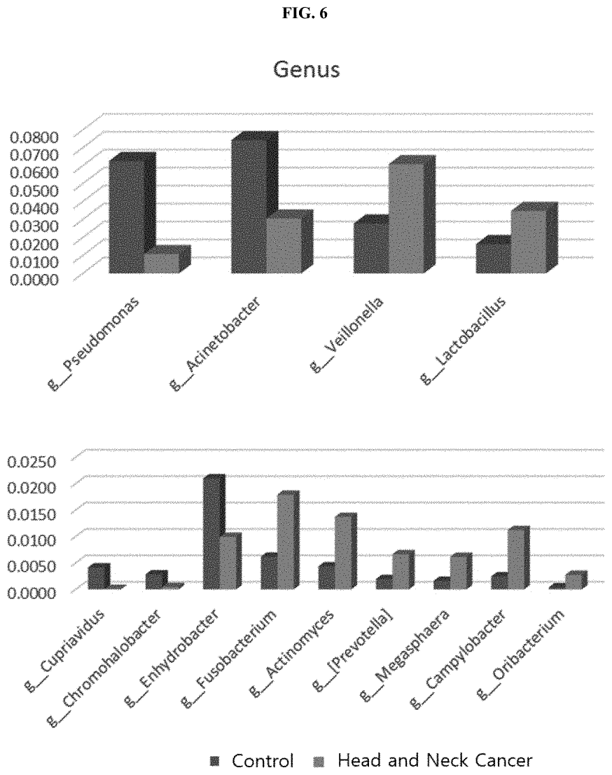

As a result of analyzing bacteria-derived EVs in saliva at a genus level, a diagnostic model developed using bacteria belonging to the genus Cupriavidus, the genus Chromohalobacter, the genus Pseudomonas, the genus Acinetobacter, the genus Enhydrobacter, the genus Lactobacillus, the genus Veillonella, the genus Fusobacterium, the genus Actinomyces, the genus Prevotella, the genus Megasphaera, the genus Campylobacter, and the genus Oribacterium as a biomarker exhibited significant diagnostic performance for head and neck cancer (see Table 6 and FIG. 6).

TABLE 6

| | | | Head and | | | | | | | |
|---|---|---|---|---|---|---|---|---|---|---|
| | Control | | Neck Cancer | | t-test | | | | | |
| Taxon | Mean | SD | Mean | SD | p-value | Ratio | AUC | Accuracy | sensitivity | specificity |
| g_Cupriavidus | 0.0041 | 0.0115 | 0.0001 | 0.0003 | 0.0000 | 0.02 | 0.78 | 0.81 | 0.98 | 0.08 |
| g_Chromohalobacter | 0.0028 | 0.0041 | 0.0004 | 0.0009 | 0.0000 | 0.15 | 0.80 | 0.82 | 0.99 | 0.10 |
| g_Pseudomonas | 0.0626 | 0.1525 | 0.0107 | 0.0116 | 0.0000 | 0.17 | 0.76 | 0.83 | 1.00 | 0.08 |
| g_Acinetobacter | 0.0742 | 0.0878 | 0.0305 | 0.0286 | 0.0000 | 0.41 | 0.71 | 0.82 | 1.00 | 0.02 |
| g_Enhydrobacter | 0.0210 | 0.0398 | 0.0099 | 0.0092 | 0.0003 | 0.47 | 0.69 | 0.82 | 1.00 | 0.02 |
| g_Lactobacillus | 0.0163 | 0.0156 | 0.0347 | 0.0451 | 0.0067 | 2.13 | 0.75 | 0.82 | 0.97 | 0.14 |
| g_Veillonella | 0.0278 | 0.0436 | 0.0608 | 0.0550 | 0.0002 | 2.19 | 0.72 | 0.81 | 0.98 | 0.08 |
| g_Fusobacterium | 0.0062 | 0.0096 | 0.0179 | 0.0273 | 0.0045 | 2.90 | 0.68 | 0.83 | 0.99 | 0.14 |
| g_Actinomyces | 0.0043 | 0.0061 | 0.0137 | 0.0121 | 0.0000 | 3.16 | 0.81 | 0.83 | 0.97 | 0.24 |
| g_[Prevotella] | 0.0019 | 0.0032 | 0.0066 | 0.0084 | 0.0003 | 3.42 | 0.74 | 0.83 | 0.98 | 0.22 |
| g_Megasphaera | 0.0016 | 0.0029 | 0.0061 | 0.0077 | 0.0002 | 3.82 | 0.77 | 0.82 | 0.96 | 0.20 |
| g_Campylobacter | 0.0025 | 0.0045 | 0.0112 | 0.0200 | 0.0037 | 4.57 | 0.75 | 0.84 | 0.99 | 0.20 |
| g_Oribacteriurn | 0.0004 | 0.0008 | 0.0027 | 0.0039 | 0.0001 | 7.35 | 0.77 | 0.85 | 0.98 | 0.28 |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

The method of providing information for diagnosing head and neck cancer through a bacterial metagenomic analysis according to the present invention may be used for predicting the risk of head and neck cancer onset and diagnosing head and neck cancer by performing a bacterial metagenomic analysis using normal individual-derived and subject-derived samples to analyze an increase or decrease in the content of specific bacteria-derived extracellular vesicles.

1. A method of providing information for diagnosing head and neck cancer, the method comprising:
(a) extracting DNAs from extracellular vesicles isolated from normal individual and subject samples;
(b) performing polymerase chain reaction (PCR) on the extracted DNA using a pair of primers comprising SEQ ID NO: 1 and SEQ ID NO: 2; and
(c) comparing an increase or decrease in content of bacteria-derived extracellular vesicles of the subject-derived sample with that of a normal individual-derived sample through sequencing of a product of the PCR.

2. The method of claim 1, wherein process (c) comprises comparing an increase or decrease in content of extracellular vesicles derived from one or more bacteria selected from the group consisting of the phylum Cyanobacteria and the phylum Fusobacteria.

3. The method of claim 1, wherein process (c) comprises comparing an increase or decrease in content of extracellular vesicles derived from one or more bacteria selected from the group consisting of the class Halobacteria, the class Chloroplast, the class Fusobacteriia, and the class Epsilonproteobacteria.

4. The method of claim 1, wherein process (c) comprises comparing an increase or decrease in content of extracellular vesicles derived from one or more bacteria selected from the group consisting of the order Halobacteriales, the order Bifidobacteriales, the order Streptophyta, the order Pseudomonadales, the order Oceanospirillales, the order Fusobacteriales, and the order Campylobacterale.

5. The method of claim 1, wherein process (c) comprises comparing an increase or decrease in content of extracellular vesicles derived from one or more bacteria selected from the group consisting of the family Pseudomonadaceae, the family Halobacteriaceae, the family Oxalobacteraceae, the family Halomonadaceae, the family Comamonadaceae, the family Lactobacillaceae, the family Paraprevotellaceae, the family Fusobacteriaceae, and the family Campylobacteraceae.

6. The method of claim 1, wherein process (c) comprises comparing an increase or decrease in content of extracellular vesicles derived from one or more bacteria selected from the group consisting of the genus Cupriavidus, the genus Chromohalobacter, the genus Pseudomonas, the genus Acinetobacter, the genus Enhydrobacter, the genus Lactobacillus, the genus Veillonella, the genus Fusobacterium, the genus Actinomyces, the genus Prevotella, the genus Megasphaera, the genus Campylobacter, and the genus Oribacterium.

7. The method of claim 1, wherein the normal individual and subject sample is saliva.

8. The method of claim 1, wherein process (c) comprises comparing an increase or decrease in content of extracellular vesicles derived from one or more bacteria selected from the group consisting of the phylum Cyanobacteria and the phylum Fusobacteria;
extracellular vesicles derived from one or more bacteria selected from the group consisting of the class Halobacteria, the class Chloroplast, the class Fusobacteriia, and the class Epsilonproteobacteria;
extracellular vesicles derived from one or more bacteria selected from the group consisting of the order Halobacteriales, the order Bifidobacteriales, the order Streptophyta, the order Pseudomonadales, the order Oceanospirillales, the order Fusobacteriales, and the order Campylobacterale;
extracellular vesicles derived from one or more bacteria selected from the group consisting of the family Pseudomonadaceae, the family Halobacteriaceae, the family Oxalobacteraceae, the family Halomonadaceae, the family Comamonadaceae, the family Lactobacillaceae, the family Paraprevotellaceae, the family Fusobacteriaceae, and the family Campylobacteraceae; or
extracellular vesicles derived from one or more bacteria selected from the group consisting of the genus Cupriavidus, the genus Chromohalobacter, the genus Pseudomonas, the genus Acinetobacter, the genus Enhydrobacter, the genus Lactobacillus, the genus Veillonella, the genus Fusobacterium, the genus Actinomyces, the genus Prevotella, the genus Megasphaera, the genus Campylobacter, and the genus Oribacterium.

9. The method of claim 8, wherein in process (c), in comparison with the normal individual-derived sample, an increase in the content of the following is diagnosed as head and neck cancer:
extracellular vesicles derived from bacteria of the phylum Fusobacteria,
extracellular vesicles derived from one or more bacteria selected from the group consisting of the class Fusobacteriia and the class Epsilonproteobacteria,
extracellular vesicles derived from one or more bacteria selected from the group consisting of the order Fusobacteriales and the order Campylobacterales,
extracellular vesicles derived from one or more bacteria selected from the group consisting of the family Lactobacillaceae, the family Paraprevotellaceae, the family Fusobacteriaceae, and the family Campylobacteraceae, or
extracellular vesicles derived from one or more bacteria selected from the group consisting of the genus Lactobacillus, the genus Veillonella, the genus Fusobacterium, the genus Actinomyces, the genus Prevotella, the genus Megasphaera, the genus Campylobacter, and the genus Oribacterium.

10. The method of claim 8, wherein in process (c), in comparison with the normal individual-derived sample, a decrease in the content of the following is diagnosed as head and neck cancer:
extracellular vesicles derived from bacteria of the phylum Cyanobacteria,
extracellular vesicles derived from one or more bacteria selected from the group consisting of the class Halobacteria and the class Chloroplast,
extracellular vesicles derived from one or more bacteria selected from the group consisting of the order Halobacteriales, the order Bifidobacteriales, the order Streptophyta, the order Pseudomonadales, and the order Oceanospirillales,
extracellular vesicles derived from one or more bacteria selected from the group consisting of the family Pseudomonadaceae, the family Halobacteriaceae, the family Oxalobacteraceae, the family Halomonadaceae, and the family Comamonadaceae, or
extracellular vesicles derived from one or more bacteria selected from the group consisting of the genus Cupriavidus, the genus Chromohalobacter, the genus Pseudomonas, the genus Acinetobacter, and the genus Enhydrobacter.

11. A method of diagnosing head and neck cancer, the method comprising:
(a) extracting DNAs from extracellular vesicles isolated from normal individual and subject samples;
(b) performing polymerase chain reaction (PCR) on the extracted DNA using a pair of primers comprising SEQ ID NO: 1 and SEQ ID NO: 2; and
(c) comparing an increase or decrease in content of bacteria-derived extracellular vesicles of the subject-derived sample with that of a normal individual-derived sample through sequencing of a product of the PCR.

12. The method of claim 11, wherein process (c) comprises comparing an increase or decrease in content of extracellular vesicles derived from one or more bacteria selected from the group consisting of the phylum Cyanobacteria and the phylum Fusobacteria.

13. The method of claim 11, wherein process (c) comprises comparing an increase or decrease in content of extracellular vesicles derived from one or more bacteria selected from the group consisting of the class Halobacteria, the class Chloroplast, the class Fusobacteriia, and the class Epsilonproteobacteria.

14. The method of claim 11, wherein process (c) comprises comparing an increase or decrease in content of extracellular vesicles derived from one or more bacteria selected from the group consisting of the order Halobacteriales, the order Bifidobacteriales, the order Streptophyta, the order Pseudomonadales, the order Oceanospirillales, the order Fusobacteriales, and the order Campylobacterale.

15. The method of claim 11, wherein process (c) comprises comparing an increase or decrease in content of extracellular vesicles derived from one or more bacteria selected from the group consisting of the family Pseudomonadaceae, the family Halobacteriaceae, the family Oxalobacteraceae, the family Halomonadaceae, the family Comamonadaceae, the family Lactobacillaceae, the family Paraprevotellaceae, the family Fusobacteriaceae, and the family Campylobacteraceae.

16. The method of claim 11, wherein process (c) comprises comparing an increase or decrease in content of extracellular vesicles derived from one or more bacteria selected from the group consisting of the genus Cupriavidus, the genus Chromohalobacter, the genus Pseudomonas, the genus Acinetobacter, the genus Enhydrobacter, the genus Lactobacillus, the genus Veillonella, the genus Fusobacterium, the genus Actinomyces, the genus Prevotella, the genus Megasphaera, the genus Campylobacter, and the genus Oribacterium.

17. The method of claim 11, wherein the normal individual and subject sample is saliva.

18. The method of claim 11, wherein process (c) comprises comparing an increase or decrease in content of extracellular vesicles derived from one or more bacteria selected from the group consisting of the phylum Cyanobacteria and the phylum Fusobacteria;
extracellular vesicles derived from one or more bacteria selected from the group consisting of the class Halobacteria, the class Chloroplast, the class Fusobacteriia, and the class Epsilonproteobacteria;
extracellular vesicles derived from one or more bacteria selected from the group consisting of the order Halobacteriales, the order Bifidobacteriales, the order Streptophyta, the order Pseudomonadales, the order Oceanospirillales, the order Fusobacteriales, and the order Campylobacterale;
extracellular vesicles derived from one or more bacteria selected from the group consisting of the family Pseudomonadaceae, the family Halobacteriaceae, the family Oxalobacteraceae, the family Halomonadaceae, the family Comamonadaceae, the family Lactobacillaceae, the family Paraprevotellaceae, the family Fusobacteriaceae, and the family Campylobacteraceae; or
extracellular vesicles derived from one or more bacteria selected from the group consisting of the genus Cupriavidus, the genus Chromohalobacter, the genus Pseudomonas, the genus Acinetobacter, the genus Enhydrobacter, the genus Lactobacillus, the genus Veillonella, the genus Fusobacterium, the genus Actinomyces, the genus Prevotella, the genus Megasphaera, the genus Campylobacter, and the genus Oribacterium.

19. The method of claim 18, wherein in process (c), in comparison with the normal individual-derived sample, an increase in the content of the following is diagnosed as head and neck cancer:
extracellular vesicles derived from bacteria of the phylum Fusobacteria,
extracellular vesicles derived from one or more bacteria selected from the group consisting of the class Fusobacteriia and the class Epsilonproteobacteria,
extracellular vesicles derived from one or more bacteria selected from the group consisting of the order Fusobacteriales and the order Campylobacterales,
extracellular vesicles derived from one or more bacteria selected from the group consisting of the family Lactobacillaceae, the family Paraprevotellaceae, the family Fusobacteriaceae, and the family Campylobacteraceae, or
extracellular vesicles derived from one or more bacteria selected from the group consisting of the genus Lactobacillus, the genus Veillonella, the genus Fusobacterium, the genus Actinomyces, the genus Prevotella, the genus Megasphaera, the genus Campylobacter, and the genus Oribacterium.

20. The method of claim 18, wherein in process (c), in comparison with the normal individual-derived sample, a decrease in the content of the following is diagnosed as head and neck cancer:
extracellular vesicles derived from bacteria of the phylum Cyanobacteria,
extracellular vesicles derived from one or more bacteria selected from the group consisting of the class Halobacteria and the class Chloroplast,
extracellular vesicles derived from one or more bacteria selected from the group consisting of the order Halobacteriales, the order Bifidobacteriales, the order Streptophyta, the order Pseudomonadales, and the order Oceanospirillales,
extracellular vesicles derived from one or more bacteria selected from the group consisting of the family Pseudomonadaceae, the family Halobacteriaceae, the family Oxalobacteraceae, the family Halomonadaceae, and the family Comamonadaceae, or
extracellular vesicles derived from one or more bacteria selected from the group consisting of the genus Cupriavidus, the genus Chromohalobacter, the genus Pseudomonas, the genus Acinetobacter, and the genus Enhydrobacter.
